(19) United States
(12) Patent Application Publication
(10) Pub. No.: US 2008/0058458 A1
(43) Pub. Date: Mar. 6, 2008

(54) PREPARATION OF DECAHALODIPHENYL ETHANE

(75) Inventors: Alvin E. Harkins, Baton Rouge, LA (US); James E. Boone, Baton Rouge, LA (US)

Correspondence Address: McGLINCHEY STAFFORD, PLLC, 4703 BLUEBONNET BLVD, BATON ROUGE, LA, 70809, US

(73) Assignee: ALBEMARLE CORPORATION, Baton Rouge, LA (US)

(21) Appl. No.: 11/840,293

(22) Filed: Aug. 17, 2007

Related U.S. Application Data
(60) Provisional application No. 60/823,862, filed on Aug. 29, 2006.

(51) Int. Cl. C07C 25/00; C08K 5/03

(52) U.S. Cl. 524/464; 570/185; 570/208

(57) ABSTRACT

This invention provides a process of preparing reaction-derived decahalodiphenylethane of high purity. The process comprises cofeeding separate feeds of
(a) diphenylethane and
(b) bromine chloride, bromine chloride and bromine, or bromine chloride and chlorine to a refluxing reaction mixture comprising bromine and at least one Lewis acid bromination catalyst so that high purity decahalodiphenylethane is formed.

### REFERENCE TO RELATED APPLICATION

This application claims the benefit and priority of U.S. Provisional Application No. 60/823,862, filed Aug. 29, 2006, the disclosure of which is incorporated herein by reference.

### TECHNICAL FIELD

This invention relates to the preparation of decahalodiphenylethane products of high purity and their use in flammable materials.

### BACKGROUND

Decabromodiphenylethane (1,2-bis(pentabromophenyl)ethane) is a time-proven flame retardant for use in many flammable macromolecular materials, e.g., thermoplastics, thermosets, cellulosic materials, and back coating applications.

Decabromodiphenylethane is presently sold as a powder derived from the bromination of 1,2-diphenylethane. Among prior processes for effecting such bromination are the bromination processes described in U.S. Pat. Nos. 6,518,468; 6,958,423; 6,603,049; 6,768,033; and 6,974,887. While it has been possible in the past to product very high purity decabromodiphenylethane, this has not been accomplished on a consistent basis. Accordingly, it would be desirable if process technology could be provided that would enable the production of highly pure decabromodiphenylethane on a consistent basis.

### SUMMARY OF INVENTION

This invention is deemed to enable production of decahalodiphenylethane products having a higher degree of halogenation and lower contents of nonabromodiphenylethanes without recourse to recrystallization or chromatographic purification steps. This invention is generally directed to the production of a product which is perhalogenated with respect to the aromatic rings of 1,2-diphenylethane. As used throughout this document, the term “decabromodiphenylethane” means 1,2-bis(pentabromophenyl)ethane.

Thus, in accordance with this invention, an embodiment provided by this invention is a process of preparing reaction-derived decahalodiphenylethane of high purity. The process comprises cofeeding separate feeds of

(a) diphenylethane and
(b) bromine chloride, bromine chloride and bromine, or bromine chloride and chlorine to a refluxing reaction mixture comprising bromine and at least one Lewis acid bromination catalyst so that high purity decahalodiphenylethane is formed.

Another embodiment of this invention is a reaction-derived product containing (i) at least 96% of decabromodiphenylethane, (ii) nonabromodiphenylethane in an amount not exceeding 0.5%, and (iii) nonabromochlorodiphenylethane in an amount of about 0.1% to about 3%.

Pursuant to this invention, it is deemed possible to prepare reaction-derived decabromodiphenylethane products containing:
A) (i) at least 96% decabromodiphenylethane, (ii) nonabromodiphenylethane in an amount not exceeding 0.5%, and (iii) nonabromochlorodiphenylethane in an amount of about 0.1% to about 3%.
B) (i) at least 97% decabromodiphenylethane, (ii) nonabromodiphenylethane in an amount not exceeding about 0.3%, (iii) nonabromochlorodiphenylethane in an amount of about 0.1% to about 3%;
C) (i) at least 99% decabromodiphenylethane, (ii) nonabromodiphenylethane in an amount not exceeding about 0.3% (iii) nonabromochlorodiphenylethane in an amount of about 0.1% to about 0.7%; and
D) (i) at least 96% decabromodiphenylethane, (ii) nonabromodiphenylethane in an amount not exceeding 0.5%, and (iii) nonabromochlorodiphenylethane in an amount of about 0.2% to about 2.5%.

These and other embodiments and features of this invention will be still further apparent from the ensuing description and appended claims.

### FURTHER DETAILED DESCRIPTION OF THE INVENTION

As used throughout this document, the term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived.” In other words, the products are directly produced in the synthesis process without use of any subsequent procedure to remove or that removes nonabromodiphenylethane from decahalodiphenylethanes.

As used throughout this document, the term “decahalodiphenylethane” refers to arperhalogenated diphenylethanes that contain only bromine or contain only bromine and chlorine on the aromatic rings. Preferably, the ethylene bridge of these compounds is not halogenated or at least no more than about 0.5 weight percent of the total product has halogen substituent(s) on the ethylene bridge. Examples of decahalodiphenylethanes include decabromodiphenylethane (Br₁₀DPE) and nonabromochlorodiphenylethane (Br₉ClDPE).

As used throughout this document, the term “high purity” means that the reaction-derived decahalodiphenylethane product comprises more than 97% decahalodiphenylethane species, with the balance consisting essentially of nonabromodiphenylethane (Br₉DPE), octabromochlorodiphenylethane (Br₈ClDPE), and/or octabromodiphenylethane (Br₈DPE), with the amount of Br₈DPE being less than the amount of Br₉DPE. Preferred reaction-derived decahalodiphenylethane product comprises at least 98% of decabromodiphenylethane, and more preferred reaction-derived decahalodiphenylethane product comprises at least 99% decabromodiphenylethane, in both cases, with the balance consisting essentially of Br₉DPE, Br₈ClDPE, and Br₈DPE and again with the amount of Br₉DPE exceeding the amount of Br₈DPE. More preferably, the reaction-derived decahalodiphenylethane product contains, at most, only a trace amount of Br₈DPE, if any. Especially preferred reaction-derived decahalodiphenyl oxide product comprises about 99% or more decahalodiphenylethane species, preferably in which nonabromodiphenylethane is present in an amount not exceeding about 0.5%. Preferably the processes of the invention form reaction-derived products which comprise (i) at least 99.5% of decahalodiphenylethane and (ii) nonabromodiphenylethane in an amount not exceeding 0.5%, preferably not exceeding 0.3%.

For the purposes of this invention, unless otherwise indicated, the % values given for decabromodiphenylethane, nonabromochlorodiphenylethane, and nonabromodiphenylethane are to be understood as being the area % values that are derived from gas chromatography analysis. A recommended procedure for conducting such analyses is presented hereinafter.

This invention enables the preparation of perhalogenated diphenylethane products that are derived from the bromination of diphenylethane with lower contents of nonabromodiphenylethane. For example, it is now deemed possible to prepare reaction-derived decabromodiphenylethane of a purity of at least 96% while having nonabromodiphenylethane in an amount of 0.5% or less. Indeed, it is deemed possible to prepare reaction-derived products that contain at least 99% decabromodiphenylethane and that contain amounts of nonabromodiphenylethane not exceeding 0.3%. More preferably, the amount of nonabromodiphenylethane will not exceed about 0.2%. These reaction products will also typically contain nonabromochlorodiphenylethane in an amount of about 0.2% to about 3%. Such products can be said to be “reaction-derived” since they are reaction determined and not the result of use of downstream purification techniques, such as recrystallization, chromatography, or like procedures. In other words, the products having such high halogenation are directly produced in the synthesis process apart from use of subsequent purification procedures as applied to the recovered or isolated products.

Various iron and/or aluminum Lewis acids can be used as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in the reaction mass. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

If desired, a suitable solvent can be included in the reaction mixture. This can be advantageous in that one can have a higher reaction temperature and possibly a lower HBr concentration in the reaction mixture thereby giving higher purity decahalodiphenylethanes. Among such suitable solvents are methylene bromide (dibromomethane) and bromoform.

In the various embodiments of this invention, 1,2-diphenylethane (also called dibenzyl or bibenzyl) is used. The term “diphenylethane” as used throughout this document means 1,2-diphenylethane unless otherwise noted. The diphenylethane can be fed as solids, but preferably the feed is in molten form or as a solution in a solvent such as methylene bromide or bromoform. To prevent freeze up in the feed conduit, diphenylethane is desirably fed at a temperature of in the range of at least about 56° C. to about 80° C. Higher temperatures can be used if desired.

Throughout this disclosure reference is often made to “bromine chloride”, a term commonly used by chemists to describe a substance made by combining bromine and chlorine. This substance is generally represented in the chemical arts by the molecular formula BrCl or Br—Cl. We wish to forestall any quibbling based on hypertechnicalities, to make note of the fact that there is evidence to indicate that “bromine chloride” itself is an equimolar mixture of elemental bromine and elemental chlorine, and further that under ordinary conditions 100% pure BrCl probably does not exist as such, but rather the equimolar mixture itself apparently exists as a mixture of about 60% BrCl, 20% Br₂, and 20% Cl₂. But whatever it is, the substance known to chemists as “bromine chloride” is what is being referred to. And reference herein to a mixture of “bromine chloride and bromine” or a mixture of “bromine chloride and chlorine” simply means that besides the equimolar mixture of bromine and chlorine known to chemists as “bromine chloride”, whatever its makeup, there is an excess amount of bromine or chlorine, respectively, over the equimolar amount of bromine and chlorine. In the practice of this invention, the use of bromine chloride or bromine chloride and bromine is preferred.

In the processes of this invention, bromine chloride (or bromine chloride and bromine, or bromine chloride and chlorine) can be used in various amounts, from significantly less than that theoretically needed to perhalogenate the diphenylethane to an excess relative to the amount theoretically needed to perhalogenate the diphenylethane. More specifically, preferred amounts of bromine chloride (or bromine chloride and bromine, or bromine chloride and chlorine) are in the range of about 30% to about 130% relative to the amount theoretically needed to perhalogenate the diphenylethane; more preferred are in the range of about 50% to about 115% relative to the amount theoretically needed to perhalogenate the diphenylethane. Particularly preferred ranges are about 50% to about 60% and about 90% to about 115% relative to the amount theoretically needed to perhalogenate the diphenylethane. Larger amounts of bromine chloride than 130% of that theoretically needed for perhalogenation are not expected to further decrease the amount of nonabromodiphenylethane in the product, while an increase in the amount of chlorine in the product is expected. Less chlorine is observed in the product when less than a stoichiometric amount of bromine chloride relative to that theoretically needed for perhalogenation is used; however, a concomitant increase in the amount of nonabromodiphenylethane has been observed. It is possible to use less than the stoichiometric amount of bromine chloride theoretically needed for perhalogenation because, as is known in the art, the bromine present in the reaction mixture acts as both a solvent and as a bromination agent for the diphenylethane.

In this connection, the total amount of halogen in the reaction mixture inclusive of the bromine initially in the reactor and that fed as bromine chloride (or bromine chloride and chlorine or bromine chloride and chlorine) is preferably at least about 15 moles per mole of diphenylethane. When the feed is diphenylethane, the reaction mixture typically will contain in the range of at least about 14 moles of total halogen per mole of diphenylethane to be fed thereto, and preferably, the reaction mixture contains in the range of about 5 to about 30 moles of total halogen per mole of diphenylethane to be fed thereto. It is possible to use more than 30 moles bromine per mole of diphenylethane but this offers no advantage.

The amount of bromine initially in the reactor (before either of the cofeeds is commenced) is generally at least about 5 moles per mole of diphenylethane to be fed, and preferably is 5 to 10 moles or more per mole of diphenylethane to be fed. Amounts of bromine at the higher end of the range are preferred. When the amount of total halogen desired is greater than the combined amount of bromine initially in the reactor and the bromine chloride (or bromine chloride and chlorine or bromine chloride and chlorine) being fed, the additional bromine can be fed as a separate feed or included in the bromine chloride feed (i.e., the bromine chloride feed will be bromine chloride and bromine).

In preferred embodiments, the diphenylethane and a portion of the bromine (not the bromine chloride, bromine chloride and bromine, or bromine chloride and chlorine) are cofed down a diptube in which the diphenylethane and the cofed bromine mix at the end of the diptube. It is particularly preferred to jet the mixed diphenylethane and bromine from the diptube into the bromine/catalyst mixture. See in this connection U.S. Pat. No. 6,958,423 (2005).

In the practice of this invention, the bromine chloride, bromine chloride and bromine, or bromine chloride and chlorine may be fed above the surface, at the surface, or below the surface of the reaction mass. It is preferred to feed the bromine chloride, bromine chloride and bromine, or bromine chloride and chlorine subsurface to the reaction mass. Subsurface feeding minimizes the possibility of splattering which can occur when, for example, liquid bromine chloride strikes the surface of the reaction mass. It is to be noted that when the term “subsurface” is used anywhere in this document, including the claims, the term does not denote that there must be a headspace above the reaction mass. For example, if the reaction mass completely fills a reactor (with equal rates of incoming and outgoing flows), the term “subsurface” means in this case that the substance being fed subsurface is being fed directly into the body of the reaction mass, the surface thereof being defined by the enclosing walls of the reactor.

The processes of this invention comprise cofeeding (a) diphenylethane and (b) bromine chloride, bromine chloride and bromine, or bromine chloride and chlorine and as separate feeds to a refluxing reaction mixture of bromine and at least one Lewis acid bromination catalyst. That the feeds of the diphenylethane and the bromine chloride, bromine chloride and bromine, or bromine chloride and chlorine are cofeeds means that there is overlap in their occurrence, i.e., the cofeeds are conducted concurrently or substantially concurrently. The cofeeds do not need to begin at the same instant in time; either feed may be commenced before the other with no materially adverse effect. Similarly, the cofeeds need not end at exactly the same instant in time; one feed or the other may be stopped before the other, again without materially adverse effect. Interruptions in either feed, or both feeds, are permissible in the practice of this invention as long as such interruptions do not have a materially adverse effect. It is recommended and preferred that the feed of diphenylethane is initiated first when the feeds are not initiated at the same time in order to minimize the extent of chlorination of the diphenylethane. It is also recommended and preferred that the feed of diphenylethane is stopped first when the feeds are not stopped at the same time, again to minimize the extent of chlorination of the diphenylethane.

The processes of this invention may be conducted at atmospheric, subatmospheric, or superatmospheric pressure. Operation at atmospheric or superatmospheric pressure is preferred; more preferable is operation at atmospheric pressure. The temperature required for refluxing to effect the halogenation will vary with the pressure and the concentrations of diphenylethane, partially halogenated diphenylethanes, nonabromodiphenylethane, and decabromodiphenylethane present in the reaction mass.

Termination of the halogenation reaction is typically effected by deactivating the catalyst with water and/or an aqueous base such as a solution of sodium hydroxide or potassium hydroxide.

The decahalodiphenylethane products produced by the processes of this invention are compositions of this invention. As mentioned above, these products are deemed to comprise at least 96% decabromodiphenylethane, nonabromodiphenylethane in an amount not exceeding 0.5%, and nonabromochlorodiphenylethane, often in an amount of about 0.1% to about 3%. Preferably, the nonabromochlorodiphenylethane is present in an amount of about 0.2% to about 2.5%. The presence of such small amounts of chlorine in the decahalodiphenylethane products is not considered deleterious.

The decahalodiphenylethane products formed in processes of this invention are white or slightly off-white in color. White color is advantageous as it simplifies the end-users task of insuring consistency of color in the articles that are flame retarded with the decahalodiphenylethane products.

The decahalodiphenylethane products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The decahalodiphenylethane products of this invention can be used in textile applications, such as in latex-based back coatings.

The amount of a decahalodiphenylethane product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any give application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % decahalodiphenylethane product of this invention. Masterbatches of polymer containing decahalodiphenylethane, which are blended with additional amounts of substrate polymer, typically contain even higher concentrations of decahalodiphenylethane, e.g., up to 50 wt % or more.

It is advantageous to use the decahalodiphenylethane products of this invention in combination with antimony-based synergists, e.g. Sb₂O₃. Such use is conventionally practiced in all decahalodiphenylethane applications. Generally, the decahalodiphenylethane products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the decahalodiphenylethane products of this invention, e.g., plasticizers, antioxidants, fillers, pigments, UV stabilizers, etc.

Thermoplastic articles formed from formulations containing a thermoplastic polymer and decahalodiphenylethane product of this invention can be produced conventionally, e.g., by injection molding, extrusion molding, compression molding, and the like. Blow molding may also be appropriate in certain cases.

### Recommended Gas Chromatographic Procedure

The gas chromatography is on a Hewlett-Packard 5890 Series II gas chromatograph equipped with a flame ionization detector, a cool on-column temperature and pressure programmable inlet, and temperature programming capability. The column is a 12QC5 HTS capillary column, 12 meter, 0.15μ film thickness, 0.53 mm diameter, part number 054657, available from SGE, Inc. (2007 Kramer Lane, Austin, Tex. 78758). Conditions were: detector temperature 350° C.; inlet temperature 70° C.; helium carrier gas at 10 mL/min.; inlet pressure 4.0 psig (ca. 1.29×10⁵ Pa), increasing at 0.25 psi/min. to 9.0 psig (ca. 1.63×10⁵ Pa) and holding at 9.0 psig until the end of the run; oven temperature 60° C. with heating at 12° C./min. to 350° C. and holding for 10 min.; and injection mode of cool on-column. Samples were prepared by dissolving, with warming, 0.003 grams in 10 grams of dibromomethane and injection of 2 microliters of this solution. The integration of the peaks was carried out using Target Chromatography Analysis Software from Thru-Put Systems, Inc. (5750 Major Blvd., Suite 200, Orlando, Fla. 32819; currently owned by Thermo Lab Systems). However, other and commercially available software suitable for use in integrating the peaks of a chromatograph may be used.

The following examples are presented for purposes of illustration, and are not intended to impose limitations on the scope of this invention.

### EXAMPLE 1

To a 250 mL pressure bottle are added 274.3 g of Br₂ and 105.5 g of Cl₂ (2.97 equivalents of BrCl, 10% excess). The pressure bottle is equipped with a ⅛-inch (outer diameter) diptube. A reactor is configured from a 1-liter Morton flask with a mechanical stirrer, thermocouple, Friedrich condenser (2° C. cooling water on condenser), 1/32-inch (inner diameter) diptube (for diphenylethane feed), and ⅛-inch (outer diameter) diptube (for BrCl feed), and heated with a heating mantle.

The reactor is charged with 3.16 g of AlCl₃ and 711 g of bromine. Diphenylethane (49.4 g, 0.271 mol) and the Br₂/Cl₂ mixture (BRCl) are cofed to the reactor during 124 minutes at 55° C. to 57° C. The rate of addition is at a proportion of about 8.2 g Br₂/Cl₂ mixture (BrCl) per gram of diphenylethane, such that the addition of both is completed at the same time. The mixture in the reactor is refluxed for 10 minutes after the cofeeds have ended, and deionized H₂O is added. The reactor is set for distillation. The halogen (mostly Br₂, but also comprised of BrCl and C1₂) is distilled off. When most of the halogen is gone, more deionized water is added. The remaining halogen is distilled off to 100° C. The remaining mixture is cooled to 60° C., and 30 mL of 25% NaOH is added to pH 13-14. The resultant mixture is filtered and washed well with deionized water. A sample is subjected to GC analysis. The sample is oven dried.

### EXAMPLE 2

Example 1 is repeated, with the following differences. A Vigreux column is placed between the reactor and the condenser. The amounts of the reagents are 302 g of Br₂ and 53.1 g of Cl₂ in the pressure bottle, 3.4 g of AlCl₃ and 698 g of bromine charged to the reactor, and 51.0 g of diphenylethane. Diphenylethane (2 grams) is added to the reactor before the BrCl addition is begun, after which the diphenylethane and BrCl are added at rates such that addition of both is completed at about the same time. Reaction temperature is 56° C. throughout the additions. The mixture is refluxed 4 minutes longer, and then is worked up as in Example 1.

The use of the term “concurrent” does not exclude the possibility of inconsequential interruptions taking place during the cofeeds, provided that the time intervals are of sufficiently short duration to cause no material adverse effect upon the overall process. Nor does the term “concurrent” imply that the cofeeds must start at exactly the same moment in time.

A feature of this invention is the concurrent cofeeding referred to above. It is again to be emphasized that the term “concurrent” does not imply that the cofeeds must start at exactly the same time or that they must stop at exactly the same moment in time. It should also be understood that while the concurrent cofeeds are preferably continuous concurrent feeds, slight interruptions in one or both feeds are acceptable provided that the duration of the interruption is sufficiently small as to cause no material disruption in the reaction. Thus as used herein, the terms “concurrent” and “continuous” should be understood to embrace the minor departures just referred to. Naturally, those skilled in the art will strive to utilize concurrent cofeeds with as little nonconcurrence as possible. Likewise, those skilled in the art will of course seek to maintain the cofeeds continuously with as few interruptions as possible under the given circumstances in which the operation is being conducted.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

That which is claimed is:

1. A process of preparing reaction-derived decahalodiphenylethane of high purity, which process comprises cofeeding separate feeds of
(a) diphenylethane and
(b) bromine chloride, bromine chloride and bromine, or bromine chloride and chlorine to a refluxing reaction mixture comprising bromine and at least one Lewis acid bromination catalyst so that high purity decahalodiphenylethane is formed.

2. A process as in claim 1 wherein (b) is bromine chloride or bromine chloride and bromine.

3. A process as in claim 1 wherein said process is conducted at atmospheric pressure.

4. A process as in claim 1 wherein said feeding is subsurface to the liquid mixture.

5. A process as in claim 1 wherein said process is conducted at atmospheric pressure, and wherein said feeding is subsurface to the liquid mixture.

6. A process as in claim 1 wherein (b) is bromine chloride or bromine chloride and bromine, and wherein said feeding is subsurface to the liquid mixture.

7. A process as in claim 1 wherein (b) is in an amount of about 30% to about 130% relative to the amount theoretically needed to perhalogenate (a).

8. A process as in claim 1 wherein (b) is in an amount of about 50% to about 115% relative to the amount theoretically needed to perhalogenate (a).

9. A process as in claim 1 wherein (b) is bromine chloride or bromine chloride and bromine, wherein said feeding is subsurface to the liquid mixture, and wherein (b) is in an amount of about 50% to about 115% relative to the amount theoretically needed to perhalogenate (a).

10. A reaction-derived product containing (i) at least 96% decabromodiphenylethane, (ii) nonabromodiphenylethane in an amount not exceeding 0.5%, and (iii) nonabromochlorodiphenylethane in an amount of about 0.1% to about 3%.

11. A product as in claim 10 containing (i) at least 97% decabromodiphenylethane and (ii) nonabromodiphenylethane in an amount not exceeding about 0.3%.

12. A product as in claim 10 containing (i) at least 99% decabromodiphenylethane and (ii) nonabromodiphenylethane in an amount not exceeding about 0.3%.

13. A product as in claim 10 containing in the range of about 0.2% to about 2.5% nonabromochlorodiphenylethane.

14. A flammable macromolecular material containing a flame retardant amount of a reaction-derived product of any of claims 10-13.

15. A material as in claim 14 wherein the macromolecular material is a thermoplastic polymer, a thermoset polymer, or a latex-based coating.
